(19) United States
(12) Patent Application Publication
(10) Pub. No.: US 2021/0170128 A1
(43) Pub. Date: Jun. 10, 2021

(54) GUARD APPARATUS AND METHOD FOR LARYNGEAL MASK AIRWAY

(71) Applicant: Alan Ringel, Carmel, IN (US)

(72) Inventor: Alan Ringel, Carmel, IN (US)

(21) Appl. No.: 17/254,961

(22) PCT Filed: Jun. 25, 2019
(86) PCT No.: PCT/US2019/038951

§ 371 Date: Dec. 22, 2020

Related U.S. Application Data
(60) Provisional application No. 62/693,534, filed on Jul. 3, 2018.

(51) Int. Cl. A61M 16/04

(52) U.S. Cl. CPC A61M 16/0493; A61M 2250/00

(57) ABSTRACT

A guard positionable around an airway tube. The guard has a body having a through hole defined there through, an outer surface, and a plurality of valleys defined along the outer surface. The guard is positionable within a user's mouth and at least one valley of the plurality of valleys partially receives a tooth of the user to maintain the position of the guard along the airway tube.

### CROSS-REFERENCE TO RELATED APPLICATIONS

This application is a national phase entry of International Application No. PCT/US19/38951 filed Jun. 25, 2019 and which claims the benefit of U.S. Provisional Application No. 62/693,534 filed on Jul. 3, 2018, the contents of which are hereby incorporated herein by reference in their entirety.

### TECHNICAL FIELD

This disclosure relates to an accessory for a Laryngeal Mask Airway (“LMA”) device and more specifically to an accessory that prevents a patient from obstructing a tube of the LMA.

### BACKGROUND

An LMA device is frequently used in the medical field to selectively provide air to a patient's lungs. The LMA device typically has a tube with an inflatable mask or cuff at one end, the tube being inserted in the patient's mouth so that one end is located in the hypopharynx and so that the mask forms a seal in this region with the surrounding tissue.

The LMA device provides life-sustaining air to the lungs when properly placed in the patient. Often, the tube connected to the cuff is ultimately fluidly coupled to an air source that provides air to the patient's lungs at intervals and volumes required to provide proper oxygen to the patient. The oxygenated air is directed through the patient's mouth via the tube and into the lungs through the cuff. Accordingly, the tube passes through the patients mouth and between the upper and lower front teeth of the patient.

The tube of the LMA device is frequently constructed of a pliable material to allow the tube to be properly positioned within the patient. Further, the tube of the LMA device frequently causes discomfort in a patient that becomes cognizant of the use of the LMA device. More specifically, the tube frequently contacts portions of the tongue, inner mouth, and throat. As a result of this contact with the tube among other reasons, patients often bite down on the tube of the LMA device when they experience discomfort.

Further, the pliable construction of the tube makes it susceptible to closure when the patient bites on the tube. The patient may bite the pliable tube with sufficient force to substantially block the airway between the air source and the patient's lungs. Blocking the tube and airway can be detrimental to the patient when insufficient amounts of oxygenated air are provided to the patient's lungs.

Accordingly, there is a need for a guard apparatus that can be quickly positioned around the tube of an LMA device to ensure the tube is not closed if the patient bites down while the LMA device is in place in the patient.

### SUMMARY

One embodiment is a guard positionable around an airway tube. The guard has a body having a through hole defined there through and an outer surface and a plurality of valleys defined along the outer surface. The guard is positionable within a user's mouth and at least one valley of the plurality of valleys partially receives a tooth of the user to maintain the position of the guard along the airway tube.

In one example of this embodiment, the body is defined by a first side and a second side that are coupleable to one another. In one aspect of this example, the first side and the second side are coupled to one another with a hinge. Further, the first side defines one or more fastener that includes an expanded head and the second side defines a hole to receive the expanded head. The fastener does not alter the outer surface.

Another example of this embodiment has a wall defined along the through hole and formed of a different material than the outer surface. In one aspect of this example, the wall is formed of a hard plastic or metal and the outer surface is formed of a softer material than the wall. In part of this aspect, the outer surface is formed of plastic, PVC, or rubber.

In yet another example of this embodiment, the body has a wide end and a narrow end, the wide end having a greater outer width than the narrow end.

In another example, the plurality of valleys define a helical pattern about the body in the outer surface.

In yet another example of this embodiment, the body tapers inwardly between a wide end and a narrow end and the plurality of valleys define a helical pattern about the body in the outer surface.

Another embodiment of this disclosure is a guard for an airway tube. The guard has a body defining an outer surface and a through hole and a wall defined along the through hole and coupled to the body. The wall is formed of a rigid material and resists deformation caused by a biting force on the body and the outer surface is formed of a textured material that at least partially deforms responsive to the biting force.

In one example of this embodiment, the outer surface has a plurality of peaks defined therein. The plurality of peaks are defined in a helical pattern about the body. Further, the helical pattern is tapered between a wide end and a narrow end of the body.

Another example of this embodiment has a seam defined through the outer surface to the through hole, a fastener defined along a first surface of the seam, the fastener having an expanded head, and a hole defined along a second surface of the seam and sized to receive the expanded head. In one aspect of this example, the body is deformable to create a gap along the seam sufficiently wide to receive the airway tube within the through hole.

Yet another embodiment is a guard apparatus for an airway tube of a laryngeal mask airway device. The guard apparatus has a body having a first half and a second half and defining a through hole along a central axis there through, a wall coupled to the body along the through hole and formed of a material that is more rigid than the body, a hinge coupling the first half to the second half in a hinged configuration to allow the guard apparatus to be positioned around the airway tube, and a plurality of peaks and valleys defined in an outer surface of the body in a helical pattern.

In one example of this embodiment, the body is formed from a plastic, PVC, or rubber material.

### DESCRIPTION OF THE DRAWINGS

The above-mentioned aspects of the present disclosure and the manner of obtaining them will become more apparent and the disclosure itself will be better understood by reference to the following description of the embodiments of the disclosure, taken in conjunction with the accompanying drawings, wherein:

FIG. 1 is one embodiment of a typical LMA device;

FIG. 2 is an elevated perspective view of a guard apparatus for the LMA device of FIG. 1;

FIG. 3 is a side view of the guard apparatus of FIG. 2;

FIG. 4 is a lowered perspective view of the guard apparatus of FIG. 2;

FIG. 5 is a different side view of the guard apparatus of FIG. 2;

FIG. 6 is a bottom view of the guard apparatus of FIG. 2;

FIG. 7 is a top view of the guard apparatus of FIG. 2;

FIG. 8 is a top view of the guard apparatus of FIG. 2 in an open orientation;

FIG. 9 is a side view of the guard apparatus in the orientation of FIG. 8;

FIG. 10 is a different side view of the guard apparatus in orientation of FIG. 8;

FIG. 11 is an elevated perspective view of another embodiment of a guard apparatus;

FIG. 12 is a bottom view of the guard apparatus of FIG. 11;

FIG. 13 is a different perspective view of the guard apparatus of FIG. 11

FIG. 14 is a top view of the guard apparatus of FIG. 11;

FIG. 15 is a side view of the guard apparatus of FIG. 11 in an open orientation;

FIG. 16 is an elevated perspective section view of another embodiment of a guard apparatus; and

FIG. 17 is a side view of the guard apparatus of FIG. 16.

Corresponding reference numerals are used to indicate corresponding parts throughout the several views.

### DETAILED DESCRIPTION

The above-mentioned aspects of the present application and the manner of obtaining them will become more apparent and the teachings of the present application itself will be better understood by reference to the following description of the embodiments of the present application taken in conjunction with the accompanying drawings.

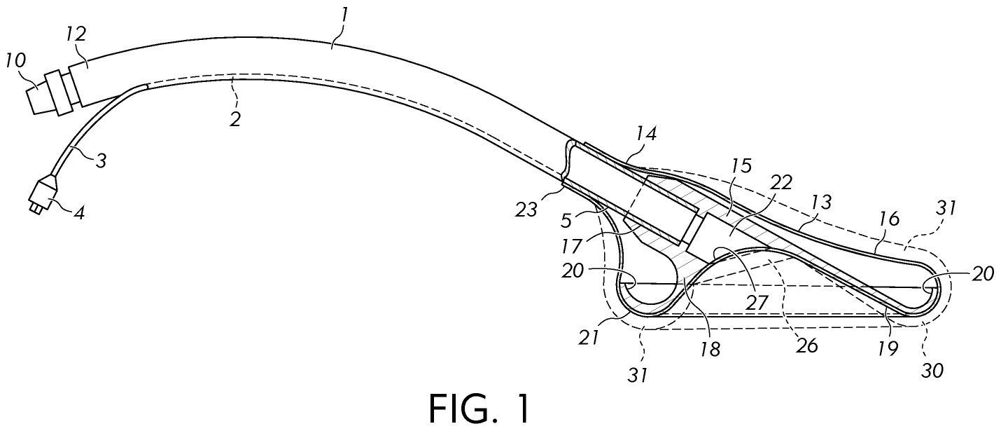

One non-exclusive example of an LMA device is shown and described in U.S. Pat. No. 5,871,012. More specifically, referring to FIG. 1, one embodiment of an LMA device 100 has a bendable tube 1 made of a plastics material, such as PVC or the like, with a coupling 10 at its machine end 12. The tube 1 may be curved along its length and has a mask portion 13 at its patient end 14. The tube 1 may be extruded with an inflation lumen 2 within its wall. The lumen 2 is connected towards the machine end of the assembly to an inflation line 3 with an inflation indicator and connector 4. The opposite, patient end of the inflation lumen 2 opens into the mask portion 13, through a hole 5 formed in the outside of the tube 1.

The mask portion 13 comprises a mount member 15 and a flexible bag member 16. The mount member 15 is molded from a bendable plastics material, such as PVC, and has a hollow cylindrical sleeve 17 at its rear end, in which the forward, patient end 14 of the tube 1 is inserted and joined. The forward, patient end 18 of the mount member 15 is of an inverted dish shape, with a generally elliptical or egg-shape outline and with a concave recess 19. The peripheral edge 20 of the mount member 15 is curved rearwardly to form a convex peripheral forward surface 21. A bore 22 extends forwardly through the mount member 15, as a continuation of the bore through the sleeve 17, and opens into the rear part of the recess 19.

The material of the bag 16 within band 26, overlying the opening of the bore 22 is cut away to form a hole 27 in the bag 16 that provides access to the bore in the mount. The hole 27 could have a series of holes or slits to prevent entry of the epiglottis. The bag 16 provides an inflatable cuff at the forward end of the assembly.

The assembly is introduced to the patient in the usual way with the cuff deflated and conforming generally to the contours of the mount 15, as shown by the solid lines in FIG. 1. When correctly positioned, a syringe or the like is connected to the connector 4 and a measured volume of air is supplied via the inflation line 3 and lumen 2 to the space between the outer surface of the mount 15 and the inner surface of the bag 16. This causes the bag 16 to inflate and distend away from the mount 15. When fully inflated, the bag 16 takes up the shape shown by the broken lines in FIG. 1.

The material of the bag 16 inflates away from the forward surface of the edge 20 of the mount 15 to form an annular conformable cushion 30 around the forward face of the patient end of the assembly. The bag 16 also inflates at the rear of the mount 15 to form a rear cushion 31. The forward cushion 30 provides a seal around the opening of the larynx by conforming to the shape of the anatomy in this region. The rear cushion 31 engages the pharyngeal wall to prevent rearward displacement of the assembly and may also gently urge the forward cushion 30 into sealing engagement.

While a specific LMA device 100 is shown and described herein, the teachings of this disclosure apply to any tubing that may be inserted through a patient's mouth. More specifically, the guard apparatus 200 described herein can be applied to any type of tubing that passes through a patient's mouth that may be susceptible to being bitten or otherwise obstructed by the patient. Further still, while this disclosure discusses primarily a human patient, the guard apparatus 200 disclosed herein may equally apply to animals as well.

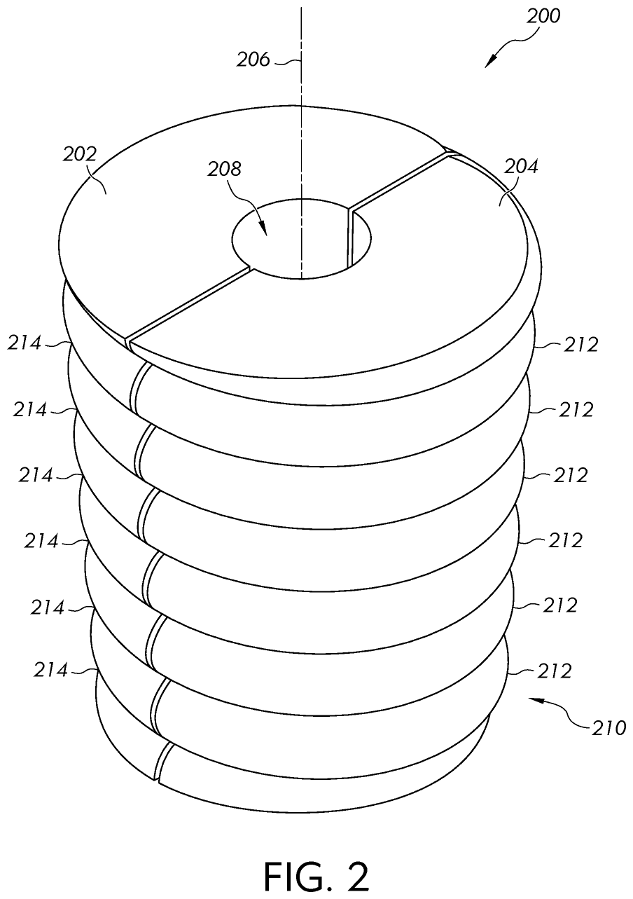

Referring now to FIG. 2, one embodiment of a guard apparatus 200 is illustrated. The guard apparatus 200 may have a first half 202 and a second half 204 that can be selectively positioned around an axis 206. Further, a through hole 208 may be defined along the axis 206 by the first and second halves 202, 204 when they are in the closed orientation of FIG. 2. In one aspect of this disclosure, the through hole 208 may be sized to be the same, or slightly larger than, the diameter of the tube 1 of the LMA device 100. Further still, in other embodiments the diameter of the through hole 208 is slightly less than the diameter of the tube 1 so that guard apparatus 200 frictionally engages the tube 1 when positioned there around.

The guard apparatus 200 may also have a textured outer surface 210. The outer surface 210 may have a plurality of peaks 212 adjacent to a plurality of valleys 214 that allow the user's teeth to maintain the positioning of the guard apparatus 200 once placed there between. More specifically, the valleys 214 may be sized so the user's teeth can become at least partially positioned therein when the LMA device 100 placed in the patient and the tube 1 is passed through the guard apparatus 200. In this orientation, the pressure applied on the guard apparatus 200 with the patient's teeth is used to maintain the position of the guard apparatus 200 around the tube 1 and between the patient's upper and lower front teeth.

The present disclosure illustrates the peaks and valleys 212, 214 as having a substantially rounded outer surface, however, this disclosure considers other forms as well. More specifically, the peaks 214 may extend along a substantially straight direction from the corresponding valleys 212. In this configuration, each of the peak and valley 212, 214 cross-section may be triangular in shape. Further still, the peaks and valleys 212, 214 may be formed from substantially ninety-degree bends in the outer surface 210. In this configuration, the peaks and valleys 212, 214 may be formed of stair-step like transitions. Accordingly, this disclosure considers the outer surface 210 as having any geometric shape that can define a valley portion and a peak portion.

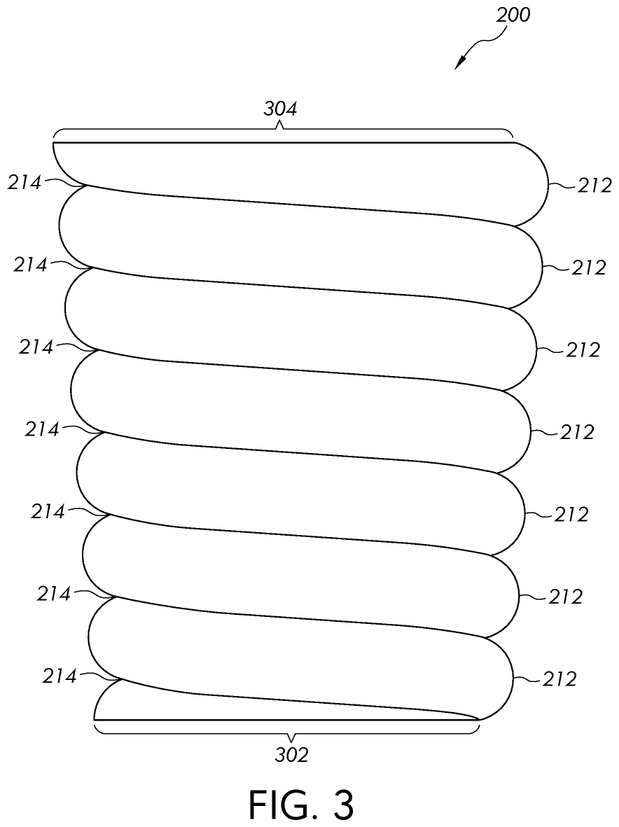

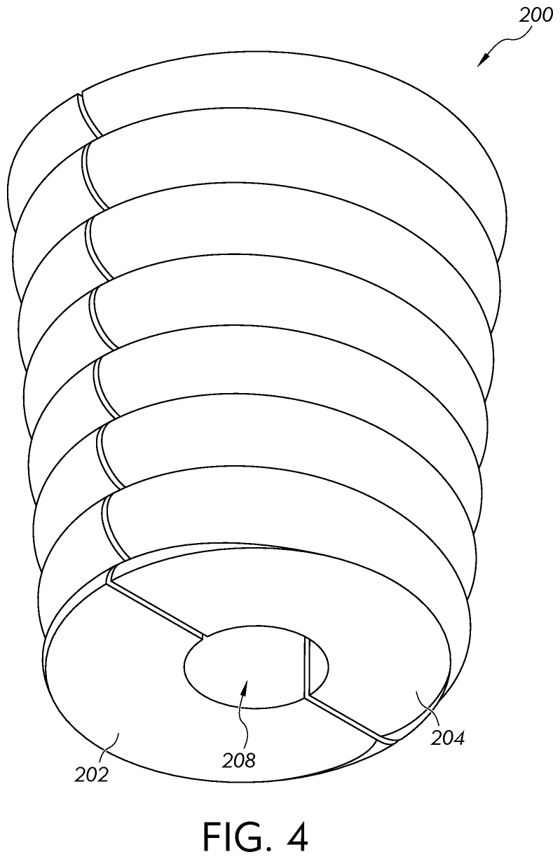

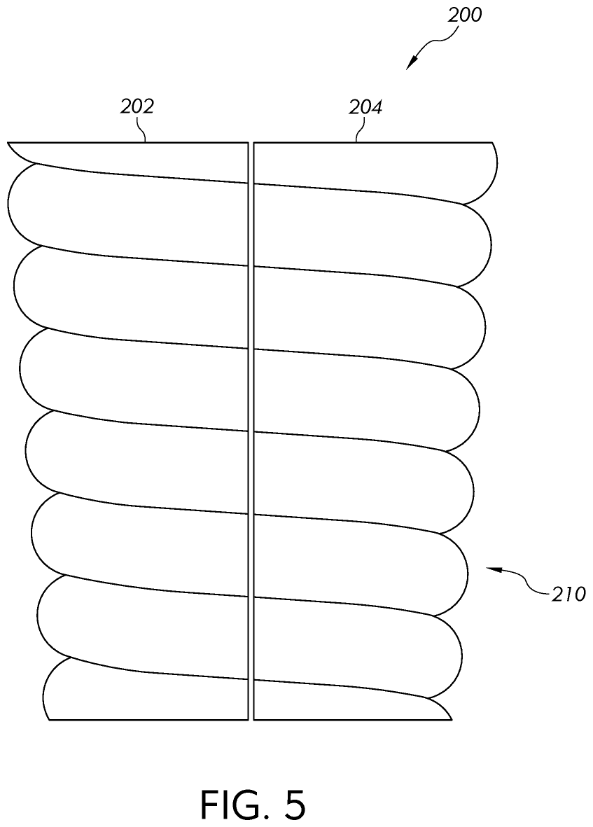

Referring now to FIG. 3, another aspect of the outer surface 210 is illustrated. More specifically, the plurality of peaks 212 and valleys 214 may be comprised of a substantially helical pattern about the axis 206. In one non-exclusive example, the plurality of peaks 212 and valleys 214 may be defined in a thread-like pattern on the outer surface 210. In this configuration, once the guard apparatus 200 is placed between the patient's teeth, the guard apparatus 200 may be rotated about the axis 206 to reposition the guard apparatus 200 within the patient's mouth.

The outer surface 210 may also taper inwardly along the length of the guard apparatus 200. More specifically, the guard apparatus 200 may have a narrow end 302 and a wide end 304. The wide end 304 may have an outer width that is greater than the outer width of the narrow end 302. Accordingly, the outer surface 210 tapers inwardly from the wide end 304 towards the narrow end 302 along the length of the guard apparatus 200.

In one aspect of this disclosure, the helical pattern of the outer surface 210 and the tapered shape between the wide end 304 and the narrow end 302 may allow the guard apparatus 200 to be implemented in patients with differing anatomy. More specifically, some patients may naturally open their mouth a width that is consistent with the width of the narrow end 302. In patients with a narrow mouth opening, the guard apparatus 200 may be placed in the patient's mouth so only the portion of the guard apparatus 200 proximate to the narrow end 302 is positioned between the patient's teeth. As one non-exclusive example, the narrow end 302 may be sized to correspond with children's mouths.

Alternatively, if the patient's anatomy naturally creates a wider opening between the patient's teeth, in adults for example, the guard apparatus 200 may be placed between the patient's teeth until a portion of the guard apparatus 200 proximate to the wide end 304 is positioned between the patient's teeth. In this configuration, the guard apparatus 200 may still contact the patient's teeth even though there is a wide gap there between. Accordingly, in one aspect of this disclosure, the tapered dimension of the guard apparatus 200 may allow it to be properly positioned in the mouths of patients with different anatomical conditions.

Further still, in one aspect of this disclosure the tapered profile and helical outer surface 210 may allow the guard apparatus 200 to be placed between the patient's teeth with sufficient friction to substantially resist further movement. More specifically, once the LMA device 100 is positioned in the patient, the guard apparatus 200 may be positioned around the tube 1. Then, the guard apparatus 200 may be positioned between the patient's teeth until they contact the outer surface 210. Next, the guard apparatus 200 may be rotated about the axis 206 while the patient's teeth travel through the valleys 214 of the outer surface 210. The engagement of the patient's teeth along the valleys 214 as the guard apparatus 200 is rotated may draw the guard apparatus 200 further into the patient's mouth, thereby expanding the gap between the patient's teeth as the wide end 304 of the guard apparatus 200 is drawn towards the patient's mouth.

In other words, the guard apparatus 200 may be rotated in a screw-like fashion to become positioned further into the patient's mouth between the patient's teeth. The guard apparatus 200 may be rotated until it has sufficiently frictionally engaged the patient's teeth so that the guard apparatus 200 does not substantially move.

In one aspect of this disclosure, the guard apparatus 200 may have a soft outer surface 210 that can contact the patient's teeth without causing substantial damage. As one non-exclusive example, the outer surface may be formed of a soft plastic, PVC, rubber, or the like. The material of the outer surface 210 may be sufficiently rigid to substantially maintain the peaks 212 and valley 214 but sufficiently soft to prevent the patient from chipping or otherwise damaging their teeth.

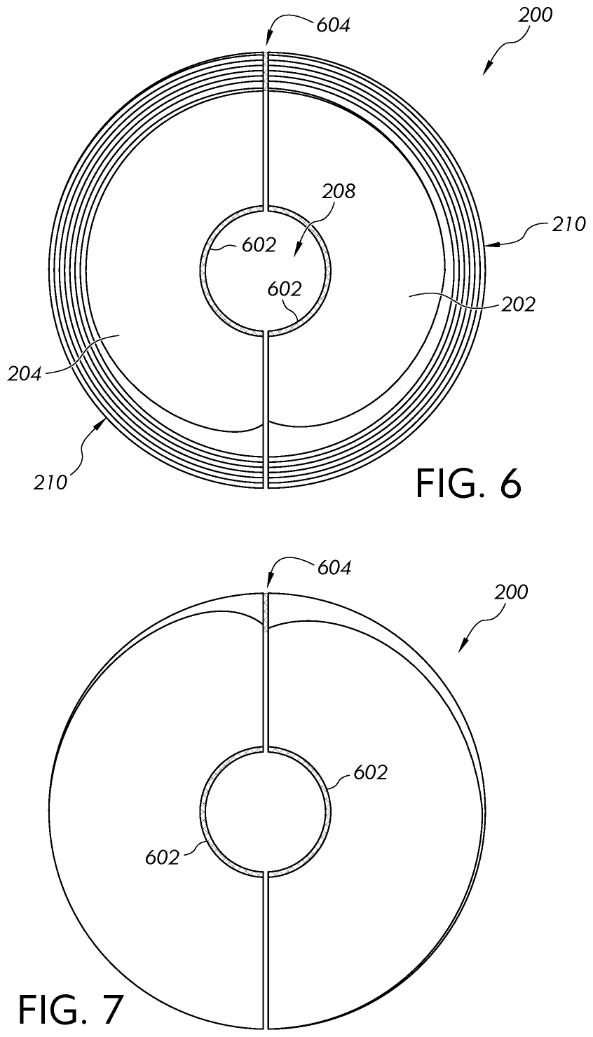

In another aspect of this disclosure, the through hole 208 may have walls formed of a substantially rigid material. More specifically with reference to FIG. 6, rigid walls 602 are illustrated about the periphery of the through hole 208. In this embodiment, the rigid walls 602 may be formed of a material that resists substantial deflection. The rigid walls 602 may be formed of a different material than the outer surface 210. As described above, the outer surface 210 may be formed of a soft material that can deflect and cushion the patient's teeth without causing damage. Alternatively, the rigid walls 602 may be rigid enough to prevent the patient from collapsing the through hole 208 with the patient's teeth. In other words, the rigid walls 602 prevent the patient from biting on the tube 1 and cutting off the airflow there through.

In one aspect of this disclosure, the rigid walls 602 are made of a hard plastic, metal, or the like while the outer surface 210 is formed of a softer plastic, rubber, or the like material. A person skilled in the relevant art understands the many different types of materials that can be used to achieve the teachings of this disclosure and the specific examples given herein are meant as non-exclusive examples. Accordingly, any known equivalent materials that are suitable to meet the needs discussed herein are considered as well.

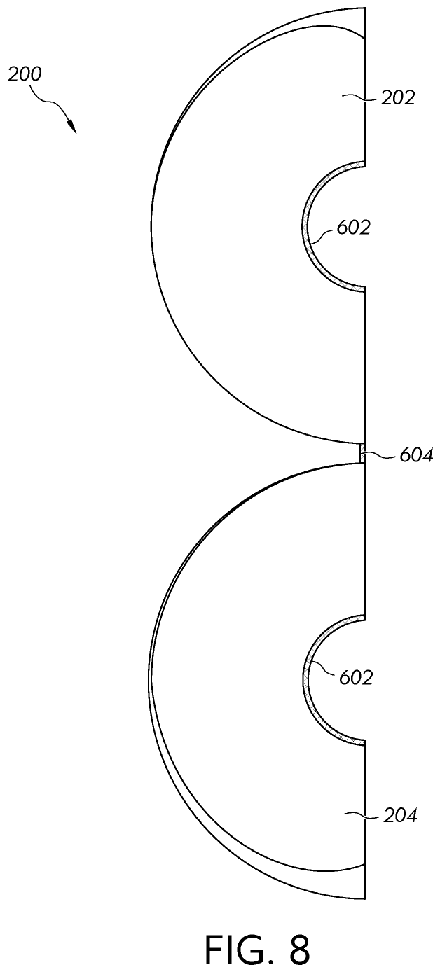

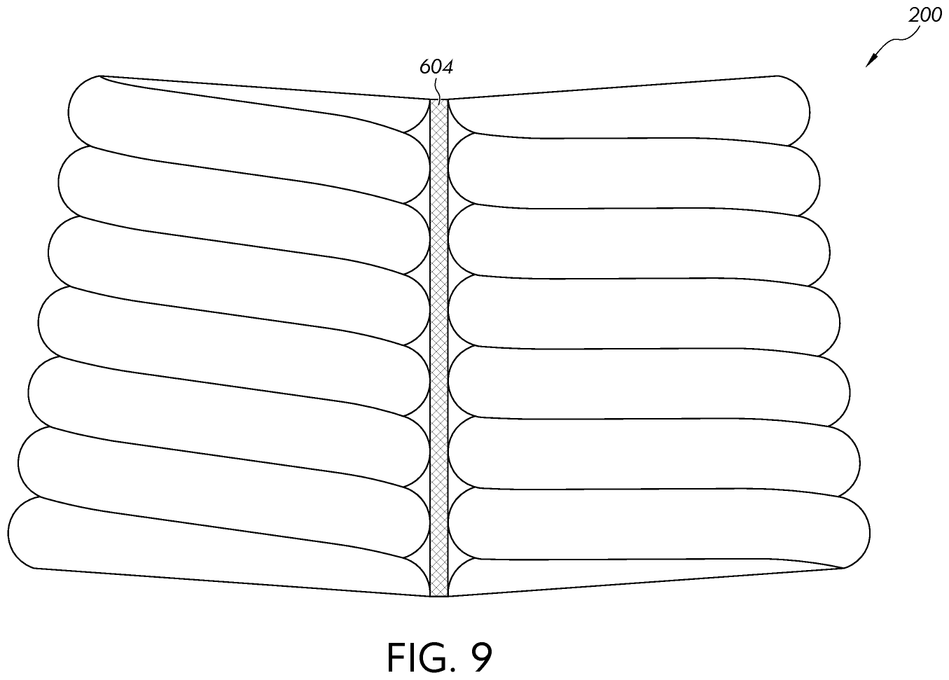

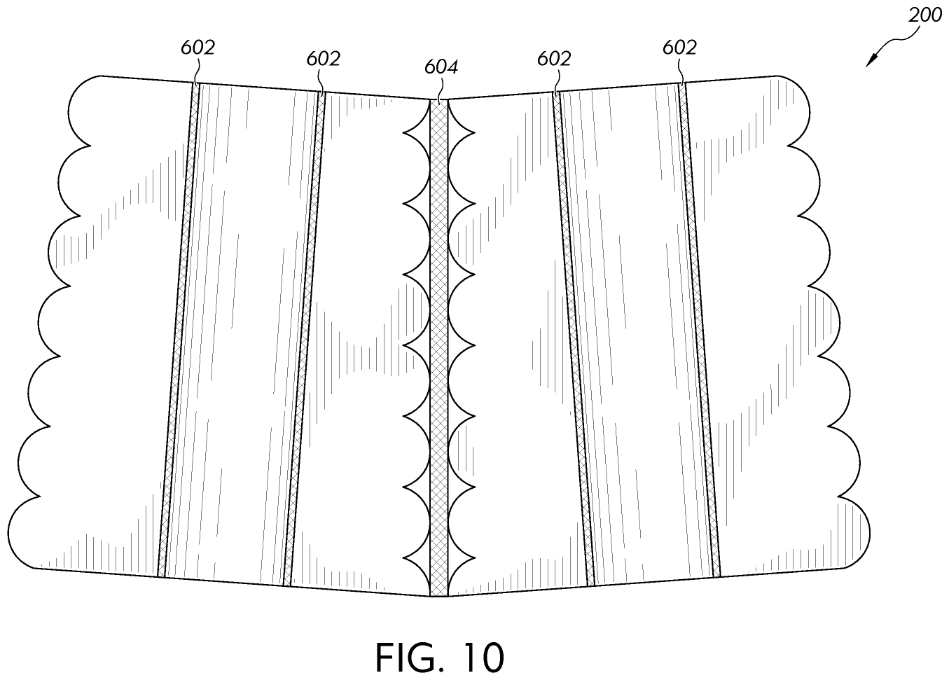

Referring now to FIGS. 6-10, a hinge 604 is illustrated on the guard apparatus 200. The hinge 604 may be defined along the outer surface 210 on one side of the guard apparatus 200 to allow the guard apparatus 200 to open sufficiently to position the tube 1 into the through holes 208. For example, the hinge 604 may allow the guard apparatus 200 to transition from the closed position of FIGS. 6 and 7 to an open position as illustrated in FIGS. 8-10.

FIGS. 8-10 illustrate the guard apparatus 200 so the first half 202 is rotated 180 degrees about the hinge 604 relative to the second half 204. In one aspect of this disclosure, the halves 202, 204 of guard apparatus 200 may pivot about the hinge 604 to be positioned around the tube 1 of the LMA device 100 after it is positioned in the patient. While FIGS. 8-10 show the halves 202, 204 rotated about 180 degrees from one another, the first and second halves 202, 204 only need to open relative to one another a sufficient angle to allow the guard apparatus 200 to be positioned around the tube 1. Accordingly, the hinge 204 does not necessarily need to be capable of allowing the first and second halve 202, 204 to pivot 180 degrees relative to one another and in other embodiments the hinge 604 may only allow the halves 202, 204 to pivot less than 180 degrees relative to one another. Further still, other embodiments of this disclosure do not have a hinge at all. Rather, in the embodiments without the hinge, the entire guard apparatus 200 may be formed with a C-shaped cross-section wherein the guard apparatus 200 may be deformed along an opening to allow the guard apparatus 200 to be positioned around the tube 1.

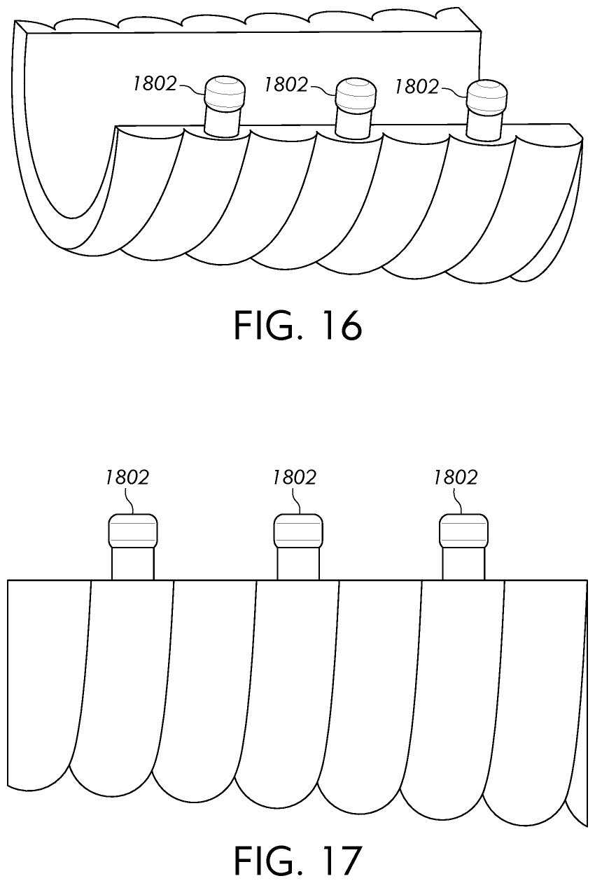

Referring now to FIGS. 16 and 17, another embodiment of this disclosure may have one or more fasteners 1802 positioned to maintain the guard apparatus 200 in the closed position. In this embodiment, the halves 202, 204 may pivot from the open position to the closed position about the hinge 604. As the halves 202, 204 become oriented in the closed position, the fasteners 1802 on the one half 202, 204 may become positioned in corresponding holes of the other half 202, 204.

In the non-exclusive example of FIGS. 16 and 17, the fasteners 1802 may have expanded heads that frictionally engage the sidewalls of the corresponding holes. Accordingly, when the halves 202, 204 are oriented in the closed position, the heads of the fasteners 1802 may slightly deform as they are forced into the corresponding holes. Once the halves are in the closed position, the heads of the fasteners 1802 may restrict the halves from separating under nominal force because of the frictional engagement of the expanded heads with the sidewalls of the corresponding holes. In other words, the fasteners 1802 are configured to maintain the guard apparatus 200 in the closed orientation unless a force separating the halves 202, 204 overcomes the frictional engagement of the fasteners 1802.

In one aspect of this disclosure, the fasteners 1802 do not interfere with the outer surface 210 or the inner surface of the through hole 208. More specifically, the fasteners 1802 may be defined between the outer surface 210 and the through hole 208 so that the patient's teeth cannot contact the fasteners 1802 when the guard apparatus 200 is in the closed position. By not allowing the fasteners 1802 to alter the outer surface 210 of the guard apparatus 200, the guard apparatus 200 may be rotated when positioned between the patient's teeth without having the fasteners 1802 contact the patient's teeth. Similarly, the fasteners 1802 may not substantially interfere with the surface of the through hole 208 to allow the guard apparatus 200 to move along the tube 1 without substantial contact from the fasteners 1802.

While one particular type of fastener is shown and described herein, this disclosure considers other types of fasteners as well. For example, clips or the like may be positioned on the end faces of the halves 202, 204 to hold them in the closed position. Further still, other embodiments may not have any fasteners at all. Accordingly, this disclosure considers many different types of fastening mechanisms.

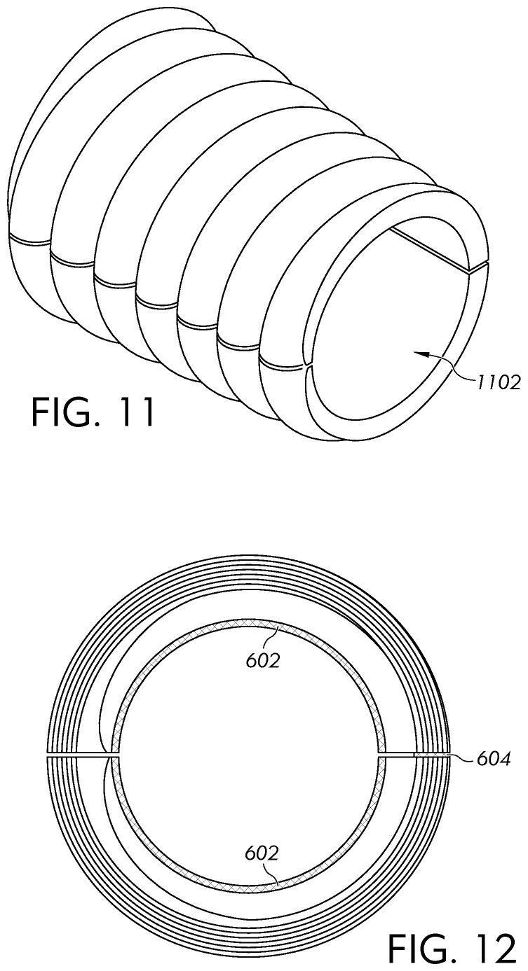

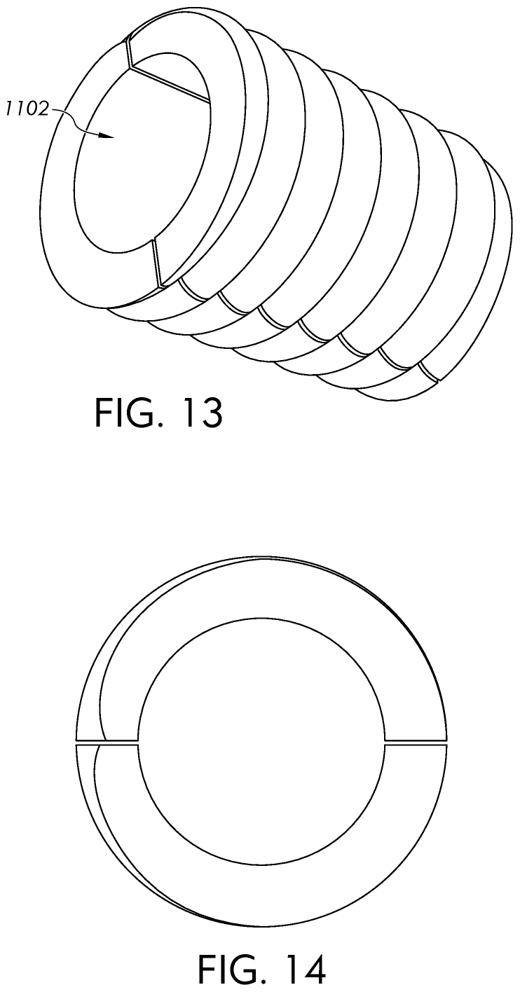

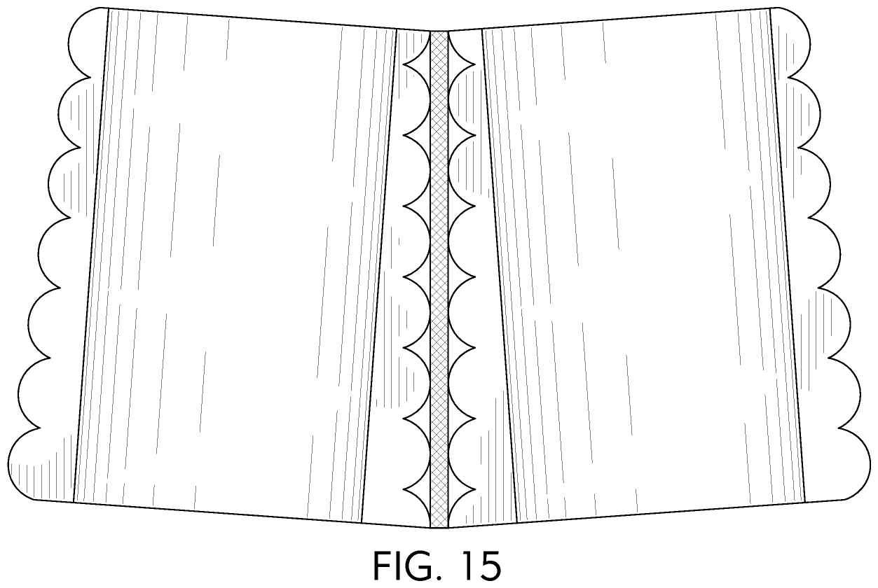

In FIGS. 11-17, a through hole 1102 may have an enlarged diameter compared to the prior embodiments. The guard apparatus of FIGS. 11-17 may otherwise have all or some of the features and components discussed herein. Further, while two different diameter through holes 208, 1102 are shown, this disclosure considers any diameter through hole that will allow the guard apparatus 200 to be placed around the tube 1.

In one aspect of this disclosure, the guard apparatus 200 may be implemented on the LMA device 100 during a medical procedure. In this example, the guard apparatus 200 may be sealed in a sterile package prior to being implemented on the LMA device 100. The LMA device 100 may be properly placed in the patient with the tube 1 routed between the patient's teeth. Next, the guard apparatus 200 may be removed from the sterile package and the halves 202, 204 may be opened relative to one another about the hinge 604 a sufficient amount to allow the guard apparatus 200 to be placed over the tube 1 with the tube 1 passing through the through hole 208. Next, the guard apparatus 200 may be moved to the closed position and any fasteners may maintain the guard apparatus 200 in the closed position. The guard apparatus 200 may then be positioned between the patient's teeth until the outer surface 210 contacts the patient's teeth. Next, the guard apparatus 200 may be rotated while contacting the patient's teeth to move the guard apparatus 200 towards the narrow end 302 or the wide end 304 to reduce or increase the force applied on the guard apparatus 200 by the patient's teeth.

While the above method of use recites placing the guard apparatus 200 around the tube 1 after the LMA device 100 is positioned in the patient, the guard apparatus 200 may be positioned around the tube 1 before the procedure all together. Accordingly, this disclosure considers placing the guard apparatus 200 around the tube 1 at any time.

Further still, while the guard apparatus 200 is described in halves herein, this disclosure also considers applications where the guard apparatus 200 is one integral component. More specifically, the guard apparatus 200 may be positioned through the tube 1 during the manufacturing process of the LMA device 100. In other words, the guard apparatus 200 may not have halves but rather require the tube 1 of the LMA device 100 be threaded through the through hole of the guard apparatus 200 prior to use.

While an exemplary embodiment incorporating the principles of the present application has been disclosed hereinabove, the present application is not limited to the disclosed embodiments. Instead, this application is intended to cover any variations, uses, or adaptations of the application using its general principles. Further, this application is intended to cover such departures from the present disclosure as come within known or customary practice in the art to which this present application pertains and which fall within the limits of the appended claims.

The terminology used herein is for the purpose of describing particular illustrative embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations).

1. A guard positionable around an airway tube, comprising:
a body having a through hole defined there through and an outer surface; and
a plurality of valleys defined along the outer surface;
wherein, the guard is positionable within a user's mouth and at least one valley of the plurality of valleys partially receives a tooth of the user to maintain the position of the guard along the airway tube.

2. The guard of claim 1, further wherein the body is defined by a first side and a second side that are coupleable to one another.

3. The guard of claim 2, further wherein the first side and the second side are coupled to one another with a hinge.

4. The guard of claim 3, further wherein the first side defines one or more fastener.

5. The guard of claim 4, further wherein the fastener includes an expanded head and the second side defines a hole to receive the expanded head.

6. The guard of claim 5, further wherein the fastener does not alter the outer surface.

7. The guard of claim 1, further comprising a wall defined along the through hole and formed of a different material than the outer surface.

8. The guard of claim 7, further wherein the wall is formed of a hard plastic or metal and the outer surface is formed of a softer material than the wall.

9. The guard of claim 8, further wherein the outer surface is formed of plastic, PVC, or rubber.

10. The guard of claim 1, further wherein the body has a wide end and a narrow end, the wide end having a greater outer width than the narrow end.

11. The guard of claim 1, further wherein the plurality of valleys define a helical pattern about the body in the outer surface.

12. The guard of claim 1, further wherein the body tapers inwardly between a wide end and a narrow end and the plurality of valleys define a helical pattern about the body in the outer surface.

13. A guard for an airway tube, comprising:
a body defining an outer surface and a through hole; and
a wall defined along the through hole and coupled to the body;
wherein the wall is formed of a rigid material and resists deformation caused by a biting force on the body and the outer surface is formed of a textured material that at least partially deforms responsive to the biting force.

14. The guard of claim 13, further wherein the outer surface has a plurality of peaks defined therein.

15. The guard of claim 14, further wherein the plurality of peaks are defined in a helical pattern about the body.

16. The guard of claim 15, further wherein the helical pattern is tapered between a wide end and a narrow end of the body.

17. The guard of claim 13, further comprising:
a seam defined through the outer surface to the through hole;
a fastener defined along a first surface of the seam, the fastener having an expanded head; and
a hole defined along a second surface of the seam and sized to receive the expanded head.

18. The guard of claim 17, further wherein the body is deformable to create a gap along the seam sufficiently wide to receive the airway tube within the through hole.

19. A guard apparatus for an airway tube of a laryngeal mask airway device, comprising:
a body having a first half and a second half and defining a through hole along a central axis there through;
a wall coupled to the body along the through hole and formed of a material that is more rigid than the body;
a hinge coupling the first half to the second half in a hinged configuration to allow the guard apparatus to be positioned around the airway tube; and
a plurality of peaks and valleys defined in an outer surface of the body in a helical pattern.

20. The guard apparatus of claim 19, further wherein the body is formed from a plastic, PVC, or rubber material.
